(19) United States
(12) Patent Application Publication
(10) Pub. No.: US 2005/0278008 A1
(43) Pub. Date: Dec. 15, 2005

(54) Thermal applicator device and method

(76) Inventor: Andrea Ladmer, El Cajon, CA (US)

Correspondence Address: Andrea Ladmer, 700 Chase Avenue, El Cajon, CA, 92020, US

(21) Appl. No.: 10/867,934

(22) Filed: Jun. 14, 2004

(51) Int. Cl. A61F 7/00

(52) U.S. Cl. 607/109; 607/114

(57) ABSTRACT

A thermal applicator device for applying cold or heat to a selected region of a user's body, particularly the head or face region, has at least two packs, each pack having a central pocket containing a thermal medium for heating or cooling purposes, and a peripheral rim. The peripheral rim has an opening on each side of the central pocket, and a lace is threaded successively through the openings in each pack so as to retain the packs on the lace. The packs can be heated or cooled prior to threading on the lace, then positioned on opposite sides of a user's face with the opposite ends of the lace extending upwardly and being tied together at the top of the user's head so as to retain the packs in position.

### BACKGROUND OF THE INVENTION

The present invention is directed to a thermal device and method for heating or cooling selected areas to reduce pain as a result of surgery, injury, or the like, and is particularly concerned with a device for application of heat or cold to a user's face or head area.

Many devices have been used in the past for thermal therapy in the head region. It is very common for oral surgeons to advise patients undergoing dental surgery to apply packs of frozen peas to the face over the affected region of the jaw in order to reduce pain and swelling. Such packs are not particularly comfortable, and it is difficult to maintain them in the correct position over an extended period of time.

There are numerous thermal therapy devices employing chilled or heated gel packs along with attachment devices for securing the packs to the desired region. Some of the attachment devices are difficult and relatively uncomfortable to use, and are adapted only for use on a specific region of the body. It is desirable for the gel pack to be easily removable from the attachment device for heating and cooling purposes.

U.S. Pat. No. 5,837,004 of Lavore describes a device for treatment of temporomandibular joint dysfunction which comprises a relatively large outer envelope for containing a gel pack, one face of envelope having a piece of hooking material, and a strap having an underside of pile or loop material for adhering to the hooking material. The gel pack can be removed from the outer envelope for heating or cooling purposes. When sufficiently hot or cold, the gel pack is re-inserted in the envelope and the envelope is placed over the treatment area with the hooking material facing outwardly. One end of the strap is then secured to the envelope by adhering the loop material to the hook material, and the remainder of the strap is extended under the chin and around the head to adhere to the opposite end of the envelope in the same way. This attachment means is relatively difficult to use and may result in slipping of the gel pack from the desired location.

U.S. Pat. No. 5,020,536 of Keen describes another postoperative therapy apparatus in which a cold pack is secured against the face by a complicated system of two vertical straps and one horizontal strap. U.S. Pat. No. 5,117,823 of Weiss et al. describes an ice cooling medical device in which ice pockets are slidably attached to an adjustable strap for securing them in place about a wearer's body. Fastening means are provided at the ends of the strap, such as fiber hook and loop material. Each ice pocket comprises an outer plastic bag and an inner plastic bag, with the inner bag containing the cooling medium, and the outer bag having two adjacent slits through which the strap is threaded. The outer bag also has an opening for removal of the inner bag when it is to be heated or cooled. The strap has a strip of double-sided adhesive tape at the location of each bag in order to hold the bag in place after positioning over the desired area, further adding to the complexity of this device.

### SUMMARY OF THE INVENTION

It is an object of the present invention to provide a new and improved thermal therapy device for applying heat or cold to a selected region of a user's body.

According to one aspect of the present invention, a thermal applicator device is provided, which comprises at least two packs, each pack having a central pocket containing a thermal medium for heating or cooling purposes, and a peripheral rim, the peripheral rim having a first opening on one side of the central pocket and a second opening on the opposite side of the central pocket, and a lace having opposite ends and being threaded through the first and second openings of each pack so as to retain the packs on the lace, whereby the packs can be positioned on opposite sides of a user's face with the opposite ends of the lace extending upwardly and being tied together at the top of the user's head so as to retain the packs in position.

In an exemplary embodiment of the invention, the lace is relatively wide with an aglet or tip of reduced dimensions at each end. The width of the lace is such that it will be more comfortable when extending under the chin and tied over the head, and will not tend to cut into the user's skin. The openings in the rim of each pack may be round holes and of diameter less than the width of the lace, such that the lace is bunched up as it extends through the holes. This will tend to hold the packs in a selected position along the lace and reduce the risk of slipping, while still allowing the user to reposition the packs relatively easily by urging them along the lace. More than two packs may be threaded on the lace if heat or cold is to be applied to more than two different locations, and a single pack may be used if only one area is to be heated or cooled.

The packs can be slid off the lace easily when they are to be heated or cooled. Any suitable thermal medium may be contained in the pocket, such as water, blue ice, or the like, but a gel which remains flexible up to a relatively low temperature is used in the exemplary embodiment. Each pack can be placed in a freezer for two hours or more if cooling therapy is desired, or may be heated in a microwave if heat therapy is to be applied.

According to another aspect of the present invention, a method of applying heat or cold to selected regions of a user's face is provided, which comprises the steps of:
taking at least two packs containing a thermal material and heating or cooling the packs for an extended period of time;
threading a lace through holes on opposite sides of a first one of the packs and subsequently threading the lace through holes on opposite sides of a second one of the packs;
sliding each pack along the lace to a predetermined position in which each pack is spaced on opposite sides of the center of the lace at an approximately equal distance from the center of the lace, whereby a central region of the lace extends between the packs and opposite end portions of the lace extend from the respective packs;
placing the packs against opposite sides of the user's face over the jaw with the threaded portions of the lace facing outwardly; and
tying the ends of the lace at the top of the user's head to retain the packs in position.

The thermal therapy device of this invention involves a minimum of parts and is easy and comfortable to use. The gel packs are not enclosed in any outer envelope and can be simply slipped off the lace when they need to be heated or cooled, and slipped back onto the lace when the desired temperature is achieved. The device is inexpensive and may be provided by doctors and dentists to their patients for use after surgery and the like.

### BRIEF DESCRIPTION OF THE DRAWINGS

The present invention will be better understood from the following detailed description of an exemplary embodiment of the invention, taken in conjunction with the accompanying drawings, in which like reference numerals refer to like parts, and in which:

### DETAILED DESCRIPTION OF THE DRAWINGS

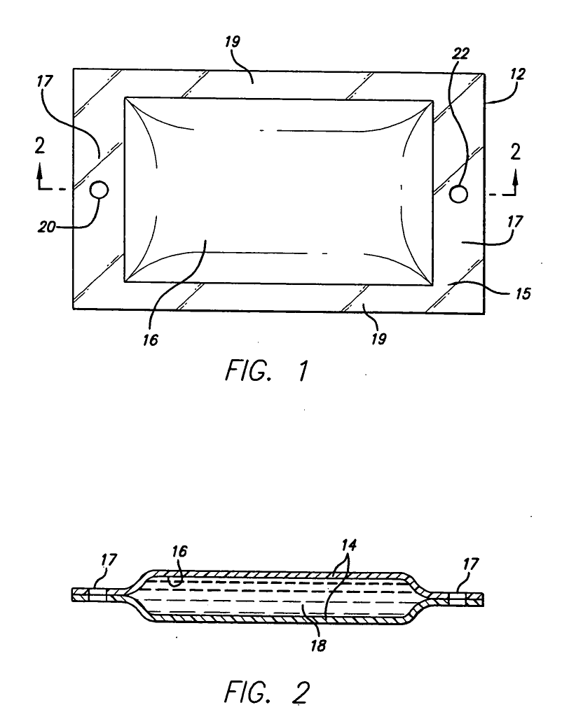

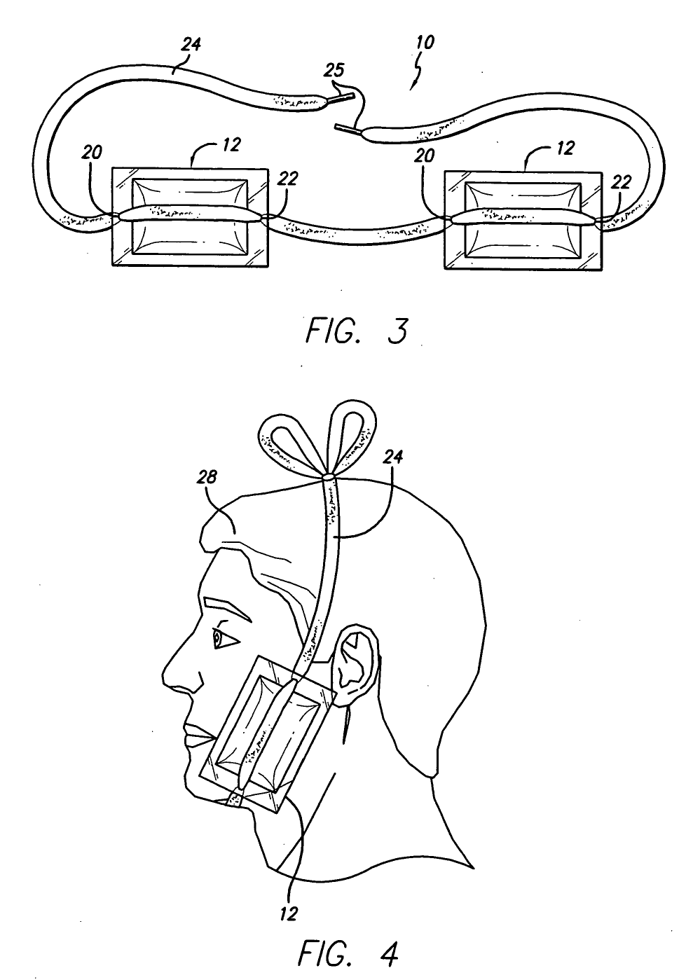

FIGS. 3 and 4 of the drawings illustrate a thermal therapy device 10 according to an exemplary embodiment of the present invention, while FIGS. 1 and 2 illustrate one of the gel packs 12 forming part of the device of FIGS. 3 and 4. Although the device of FIGS. 3 and 4 uses two gel packs, a greater or a lesser number may be used in alternative embodiments, depending on the number of areas where cold or heat is to be applied.

Each pack comprises two layers 14 of plastic material or the like cut into a rectangular shape and bonded or heat sealed together around the periphery to form a relatively wide peripheral rim 15 and a central pocket 16 containing a suitable thermal medium 18 which can be heated or cooled and which will maintain temperature for an extended period of time. The peripheral rim 15 in the illustrated embodiment is wider along the opposite side edges 17 than along the upper and lower edges 19. The width along side edges 17 may be of the order of 0.50 to 0.75 inches, while the width along the upper and lower edges 19 may be of the order of 0.25 to 0.5 inches.

Any suitable thermal medium may be used in pocket 16, but a glycerin based gel is used in the exemplary embodiment, since this will remain flexible even after cooling to relatively low temperatures. The peripheral rim 15 has a first circular opening 20 on one side 17 of the central pocket and a second circular opening 22 on the opposite side 17 of the central pocket. At least four gel packs will typically be provided, so that two can be used while two more are being heated or cooled.

The device also comprises an elongate lace or strap 24 having opposite ends. The lace is relatively wide with an aglet or tip 25 of reduced dimensions at each end. The tip 25 of the first end of the lace is threaded successively through the first and second openings 20, 22 of each pack as indicated in FIG. 3, with a portion of the lace extending over one face of each pack. It will be understood that the lace as shown in FIG. 3 is not necessarily to scale, and the lace will normally be longer to allow tying around the top of the wearer's head. In an exemplary embodiment, the lace was 48 inches long.

FIG. 4 illustrates one possible use of the device after dental surgery or the like, by tying it about a patient's head 28. Prior to use, the gel packs are placed in the freezer for a minimum of two hours. Once sufficiently cool, the packs are threaded onto the lace in the manner indicated in FIG. 3. The packs are then placed adjacent a user's cheek area on opposite sides of the face, for example above a dental surgery area, with the lace facing outwardly. The user then pulls the opposite ends of the lace upwardly to the top of the head and tying them together with a bow or knot 26 at the top of the user's head so as to retain the packs in position. If desired, a small washcloth or piece of material may be placed under the gel pack, between the pack and the user's skin.

A similar technique may be used to apply heat or cold to other regions. If heat is to be applied, the gel packs may be heated in a microwave oven. Once the packs are heated or cooled to the desired temperature, the packs are positioned appropriately on the user's head or face, or over the ears, and then tied in place using the lace.

The lace 24 is relatively wide and flat, and is made of conventional shoe lace material such as cofton or man-made material. The width of the lace may be of the order of ½″ to 1 inch. The diameter of the circular holes 20, 22 in the rim of each gel pack 12 is significantly less than the width of the lace. In an exemplary embodiment, the hole diameter is of the order of 0.25″, and may be in the range from 0.1 to 0.4 inches. This means that the lace will tend to bunch up as it is pulled through the holes, as can be seen in FIG. 3, helping to resist slipping of the gel packs from a selected position on the lace. Since no extra securing means or fasteners are required to hold the gel packs in position, the device is extremely simple, easy to use, and inexpensive. The gel packs can easily be urged along the lace to the appropriate position, and pulled off the lace when they need to be re-heated or re-cooled. However, they will not tend to slip from an adjusted position.

The thermal applicator or therapy device as described above can be used to alleviate pain and swelling associated with surgery, dental work, injury, or other trauma involving the face or jaws. It allows for hands-free delivery of thermal therapy in general or localized areas and is designed to effect relief of pain and discomfort, and alleviate swelling when used as a cold pack. The device is simple, involving a minimum of parts, and is quick and easy to use. The patient does not have to stay still in order to retain the gel packs in position, or use their hands to hold a cold or hot pack in place. Due to the inexpensive nature of the device, it could easily be provided by doctors and dentists to their patients.

Although an exemplary embodiment of the invention has been described above by way of example only, it will be understood by those skilled in the field that modifications may be made to the disclosed embodiment without departing from the scope of the invention, which is defined by the appended claims.

1. A thermal applicator device for applying cold or heat to a selected region of a user's body, comprising:
at least two packs, each pack having a central pocket containing a thermal medium for heating or cooling purposes, and a peripheral rim;
the peripheral rim having a first opening on one side of the central pocket and a second opening on the opposite side of the central pocket; and
a lace having opposite ends and being threaded successively through the first and second openings of each pack so as to retain the packs on the lace;
whereby the packs can be positioned on opposite sides of a user's face with the opposite ends of the lace extending upwardly and being tied together at the top of the user's head so as to retain the packs in position.

2. The device as claimed in claim 1, wherein the lace comprises a flat strip of pliable material of predetermined width, and each opening has a cross-sectional dimension less than the width of the strip.

3. The device as claimed in claim 2, wherein each end of the lace comprises a tip of reduced dimensions for threading through the openings.

4. The device as claimed in claim 3, wherein the width of the lace is in the range from 0.5 inches to 1 inch, and the cross-sectional dimension of the opening is in the range from 0.1 inches to 0.4 inches.

5. The device as claimed in claim 3, wherein each opening is circular.

6. The device as claimed in claim 3, wherein the width of the lace is approximately three times the cross-sectional dimension of the openings.

7. The device as claimed in claim 1, wherein the pack is rectangular and the peripheral rim has opposite sides, an upper end, and a lower end, and the opposite sides of the peripheral rim are wider than the upper and lower ends.

8. The device as claimed in claim 7, wherein the pack comprises two layers of plastic material heat sealed together around the periphery of the pack to form the peripheral rim, and the pocket is formed between the layers inside the peripheral rim.

9. The device as claimed in claim 7, wherein the width of each side of the peripheral rim is in the range from 0.50 to 0.75 inches.

10. The device as claimed in claim 9, wherein the width of the upper and lower ends of the peripheral rim is in the range from 0.25 to 0.50 inches.

11. A method of applying heat or cold to selected regions of a user's face, comprising the steps of:
taking at least two packs containing a thermal material and heating or cooling the packs for an extended period of time;
threading a lace through holes on opposite sides of a first one of the packs and subsequently threading the lace through holes on opposite sides of a second one of the packs;
sliding each pack along the lace to a predetermined position in which each pack is spaced on opposite sides of the center of the lace at an approximately equal distance from the center of the lace, whereby a central region of the lace extends between the packs and opposite end portions of the lace extend from the respective packs;
placing the packs against opposite sides of the user's face over the jaw with the threaded portions of the lace facing outwardly; and
tying the ends of the lace at the top of the user's head to retain the packs in position.
